(19) United States
(12) Patent Application Publication
(10) Pub. No.: US 2011/0312886 A1
(43) Pub. Date: Dec. 22, 2011

(54) TGF-ß AND METHODS OF TREATING OCULAR AND OTHER DISEASES

(75) Inventors: Michael E. Stern, Mission Viejo, CA (US); Christopher S. Schaumburg, Huntington Beach, CA (US); Karyn F. Siemasko, Anaheim, CA (US); Larry A. Wheeler, Irvine, CA (US)

(73) Assignee: ALLERGAN, INC., Irvine, CA (US)

(21) Appl. No.: 13/158,582

(22) Filed: Jun. 13, 2011

Related U.S. Application Data
(60) Provisional application No. 61/355,411, filed on Jun. 16, 2010.

(51) Int. Cl. A61K 38/18; A61P 43/00; A61P 27/02

(52) U.S. Cl. 514/8.9

(57) ABSTRACT

Disclosed herein is a method of treating ocular and systemic conditions by administering a TGF-β.

### CROSS-REFERENCE

This application claims the benefit of U.S. Provisional Patent Application Ser. No. 61/355,411, filed on Jun. 16, 2010, the entire disclosure of which is incorporated herein by this specific reference.

Disclosed herein are methods for treating ocular diseases, such as keratoconjunctivitis sicca, ocular cicatricial pemphigoid, blepharitis, ocular allergy, ocular infection, and diminished corneal sensitivity, and for treating systemic diseases, such as Stevens-Johnson disease and graft versus host disease, by administering to a subject transforming growth factor β.

### DESCRIPTION OF THE DRAWINGS

FIG. 1 shows an experimental protocol the inventors used to demonstrate the method of the invention. Dry Eye was induced in mice treated vehicle (PBS) or recombinant TGF-β (100 ng/5 μl/eye/BID) (Millipore #GF111 Lot #NMM1638323). After 5 days of desiccating stress CD4+ T cells were isolated and 5×10̂6 cells were adoptively transferred into nude recipient mice to evaluate the effect of TGF-β treatment on autoreactive T cell activation.

FIG. 2 shows that topical treatment with TGF-β attenuated CD4+ T cell activation following exposure to desiccating stress. Experimental Dry Eye was induced in mice treated vehicle (PBS) or recombinant TGF-β. After 5 days of desiccating stress CD4+ T cells were isolated and 5×10̂6 cells were adoptively transferred into nude recipient mice to evaluate the effect of TGF-β treatment on autoreactive T cell activation. Nude recipients of CD4+ T cells from mice exposed to desiccating stress in the presence of rTGF-β displayed a significant reduction in the number of infiltrating CD4+ T cells within the ocular surface tissues (Lid, meibomian gland—MG, conjunctiva—conj). By contrast, mice receiving only vehicle (PBS) showed marked accumulation of CD4+ T cells.

FIG. 3 shows that topical treatment with TGF-β preserved Goblet cells in nude recipient mice. Experimental Dry Eye was induced in mice treated vehicle (PBS) or recombinant TGF-β. After 5 days of desiccating stress CD4+ T cells were isolated and 5×10̂6 cells were adoptively transferred into nude recipient mice to evaluate the effect of TGF-β treatment on Goblet cells density. Nude recipients of CD4+ T cells from mice exposed to desiccating stress in the presence of rTGF-β displayed a significant preservation of the total number of Goblet cells within the conjunctiva; however, mice receiving vehicle alone displayed a dramatic loss of Goblet cells.

### DETAILED DESCRIPTION OF THE INVENTION

### Transforming Growth Factor δ

Transforming growth factors are biologically active peptides which induce anchorage-dependent, non-neoplastic cells to lose contact inhibition and to undergo anchorage-independent growth. This property is unique to transformation growth factors (“TGFs”), and it can be quantified by measuring the formation of colonies of cells in soft agar. Two classes of transforming growth factors have been identified on the basis of their relationship with epidermal growth factor. Type beta TGFs are defined by an ability to compete with 125-r-labeled EGF for binding to the EGF cell surface receptor. They are single chain, low molecular weight (Mᵣ<10,000) peptides which have been found in the conditioned medium of transformed cell lines.

In contrast, type beta TGFs do not bind to the EGF receptor, but they require EGF or TGF-α for expression of their biological activity. Although type β TGFs are present in transformed cells, they have also been detected in a variety of non-neoplastic tissues, suggesting a physiological role for these factors in modulating normal cell growth. One likely role for such growth factors is to participate in tissue repair and regeneration; TGF-β, in conjunction with EGF, is an effective promoter of wound healing in vivo.

There are five distinct homodimeric TGF-βs designated as TGF-β1, TGF-β2, TGF-β3, TGF-β4, and TGF-β5; the term “TGF-β” refers to a composition comprising 1 or more of those distinct TGF-βs. All TGF-βs are synthesized as 390 to 412 amino acid precursors that undergo proteolytic cleavage to produce the mature forms, which consist of the C-terminal 112 amino acids. In their mature, biologically active forms, TGF-β1 to 5 are acid- and heat-stable disulfide-linked homodimers of two polypeptide chains of 112 amino acids each. The complete amino acid sequences of human and simian TGF-β1 show remarkable sequence conservation, differing only in a single amino acid residue. Comparison of the amino acid sequence of human TGF-β1, TGF-β2, and human TGF-β3 has demonstrated that the three proteins exhibit in their mature forms about 70-80% sequence identity. A heterodimeric TGF-β1.2 has been isolated from porcine platelets and consists of one subunit of TGF-β1 disulfide-linked to one subunit of TGF-β2.

One can use in the method of the invention any of TGF-β1, TGF-β2, TGF-β3, TGF-β4, or TGF-β5. In one embodiment of the invention, one can use TGF-β that is isolated from human, monkey, pig, cow, mouse, or other animal. In another embodiment of the invention, one can use a TGF-β that is recombinantly produced.

Methods for isolating TGF-β from animals are well known. The isolation and purification of TGF-β from human platelets, for example, is described for example, in WO/1984/004924, the contents of which are incorporated herein by reference. The result is an isolated and purified TGF-β 25 kDa protein, designated TGF-β1, comprising a homodimer of two 12.5 kDa chains linked by disulfide bonds. Briefly, the procedure is as follows. Platelet concentrates are centrifuged (3200×g, 30 min) to remove remaining plasma proteins. The platelets are washed twice by suspension in 500-ml portions of Tris-HCl/citrate buffer, pH 7.5, and centrifuged again. Washed platelets (20-30 g wet weights) are added to a solution of acidic ethanol and immediately extracted in a homogenizer (4 ml acidic ethanol per g platelets). After incubation overnight at 4° C., precipitated proteins are removed by centrifugation, and the resulting supernatant is adjusted to pH 3 by addition of NH₄OH. Proteins and TGF activity are precipitated from the solution (overnight at 4° C.) by addition of ethanol (2 vol, 0° C.) and ethyl ether (4 vol, 0° C.). The precipitate is collected by centrifugation and suspended in 1 M acetic acid (10 ml). TGF activity is solubilized during an overnight extraction at 4° C. Centrifugation clarifies the solution; the supernatant is freeze-dried or subjected directly to gel filtration. The amount of protein in the extract may be determined by weight or by reaction with Coomassie Blue using bovine plasma albumin as reference. To purify the TGF-β, the solubilized platelet extract (10 ml in 1 M acetic acid) may be gel-filtered at a flow rate of 20 ml/h on a column (4.4×115 cm) of acrylamide gel equilibrated in 1 M acetic acid. The elution position of TGF-beta are determined by bioassay, and the fractions containing the peak of activity are pooled and freeze-dried. The residue is dissolved in 0.5 ml of 1 M acetic acid containing 8 M ultra-pure urea and gel-filtered at a flow rate of 3 ml/h on a column (1.6×85 cm) of acrylamide which is equilibrated in the sample solvent (to preclude the formation of cyanate in the solvent, the ultra-pure urea is dissolved at pH 2 in 1 M acetic acid; the resulting solution is adjusted to final conditions by addition of glacial acetic acid and water). Aliquots of selected column fractions are then tested for TGF-beta activity. Fractions containing the peak of TGF-beta activity are pooled, dialyzed against 1 M acetic acid to remove urea, and quick-frozen for storage at −20° C.

In another embodiment, one may use TGF-β isolated from pigs. WO/1988/005787, the contents of which are incorporated herein by reference, describes a method for obtaining such TGF-β, designated TGF-β2. Platelets are extracted from porcine blood by adding a solution of 1% Triton X-100, 0.2 M EGTA in a ratio of 1 ml/g, freezing to −20° C., thawing five times, centrifuging at 5-10×g for 30 minutes to remove insoluble material. Supernatant from the centrifugation is mixed with 4 parts of acidic ethanol, 50 parts of 95% ethanol, 14 parts distilled water, 1 part concentrated HCl and adjusted to pH 5.2 with concentrated ammonium hydroxide. The proteins are precipitated with two volumes of cold anhydrous ethanol and four volumes of cold anhydrous ether and allowed to stand for about 20 minutes. Precipitate is collected by centrifugation or rapid filtration through Whatman No. 1 paper and resuspended in 1 M acetic acid (about 3-4 ml per gram of tissue). Insoluble matter is removed by centrifugation at about 5-10×g for 10-30 minutes, and the supernatant is then concentrated, as by lyophilization with resuspension in 1M acetic acid. This suspension is then fractionated over successive gel filtration columns (100-200 mesh) in the absence of and then in the presence of urea. The first elution was done on a 5.0 cm×100 cm column at a flow rate of about 40 ml/h, the column having been equilibrated in 1 M acetic acid. Fractions having TGF-β activity are pooled and concentrated by lyophilization for further purification.

Methods for producing TGF-β1, TGF-β2, TGF-β3, TGF-β4, and TGF-β5 by recombinant techniques are also well known. As early as 1985, for example, U.S. Pat. No. 4,886,747, the contents of which are incorporated herein by reference, describes a method of recombinantly producing TGF-β1. The techniques are well established, and recombinantly produced TGF-β is available commercially from several suppliers.

### Methods of Treatment

The method of the invention comprises administering a TGF-β to a patient to treat ocular diseases such as keratoconjunctivitis sicca, ocular cicatricial pemphigoid, blepharitis, ocular allergy, or ocular infection, and systemic diseases such as Stevens-Johnson disease, and graft versus host disease.

In one embodiment, the method of the invention may be used to treat keratoconjunctivitis sicca. Keratoconjunctivitis sicca, as used here, refers to dry eye disease, which a patient experiences as chronic dryness of the cornea and conjunctiva. Other symptoms include a sandy-gritty feeling in the eye, burning, irritation, or a foreign-body sensation. Patients suffering from dry eye disease complain of mild to severe symptoms, and those with severe symptoms may experience constant and disabling eye irritation, and develop ocular surface epithelial disease and sight-threatening sterile or microbial corneal ulceration. Although the discomfort of keratoconjunctivitis sicca is often associated with ocular inflammation, it need not be; the only criterion is that the cause of the patient's discomfort is a deficiency in the quantity or quality of tears. Among other things, this distinguishes the dryness of keratoconjunctivitis sicca from the discomfort—sometimes perceived as dryness—in patients with other ocular disease.

In another embodiment, the method of the invention may be used to treat a patient suffering from diminished corneal sensitivity caused by refractive surgery, such as laser-assisted in situ keratomileusis (LASIK), or other trauma to the eye. Such trauma severs the corneal nerves, resulting in a state of nerve injury; this leads to discomfort, often perceived as dryness, despite that a patient may have normal tear production. Corneal sensitivity, as measured by a Cochet-Bonnet esthesiometer, usually returns to normal after a period of several months, but one can administer TGF-β to a patient to hasten that recovery.

In another embodiment, the method of the invention may be used to treat a patient suffering from ocular allergy, such as atopic keratoconjunctivitis (allergic inflammation of the eye) or vernal keratoconjunctivitis (seasonal inflammation of the eye, usually the result of allergy). In another embodiment, the method of the invention may be used to treat conjunctivitis or keratoconjunctivitis of whatever cause, such as bacterial or viral infection. In another embodiment, the method of the invention may be used to treat ocular symptoms of cicatricial pemphigoid. In another embodiment, the method of the invention may be used to treat uveitis, including anterior, intermediate, and posterior uveitis, and panuveitis.

When administering the therapeutic proteins of the invention to treat conditions of the eye, the proteins may be administered by any means that locally affects the eye, meaning that they may be administered topically, by injection, or by the implantation of a substrate or device which releases the protein into the eye. The proteins may also be administered systemically.

In another embodiment, the method of the invention may be used to treat Stevens-Johnson disease and graft versus host disease. The therapeutic proteins of the invention may be administered locally to treat Stevens-Johnson disease, such as by topically apply a cream or gel containing the protein. For both Stevens-Johnson disease and graft versus host disease the proteins may also be delivered systemically.

### Formulation

The therapeutic proteins of the present invention may be prepared for storage by mixing the antibody having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN®, PLURONICS®, or polyethylene glycol (PEG).

It is preferable that an effective amount of buffer be included to maintain the pH from about 6 to about 8, preferably about 7. Buffers used are those known to those skilled in the art, and, while not intending to be limiting, some examples are acetate, borate, carbonate, citrate, and phosphate buffers. Preferably, the buffer comprises borate. An effective amount of buffer necessary for the purposes of this invention can be readily determined by a person skilled in the art without undue experimentation. In cases where the buffer comprises borate, it is preferable that the concentration of the borate buffer be about 0.6%.

In one embodiment of the invention, a tonicity agent to be used. Tonicity agents are used in ophthalmic compositions to adjust the concentration of dissolved material to the desired isotonic range. Tonicity agents are known to those skilled in the ophthalmic art; some examples include glycerin, mannitol, sorbitol, sodium chloride, and other electrolytes. In one embodiment, the tonicity agent is sodium chloride.

In one embodiment of the invention, may a preservative to be used when the composition is intended for multiple use. There may also be reasons to use a preservative in single use compositions depending on the individual circumstances. The term preservative has the meaning commonly understood in the ophthalmic art. Preservatives are used to prevent bacterial contamination in multiple-use ophthalmic preparations, and, while not intending to be limiting, examples include benzalkonium chloride, stabilized oxychloro complexes (otherwise known as Purite®), phenylmercuric acetate, chlorobutanol, benzyl alcohol, parabens, and thimerosal. Preferably, the preservative is benzalkonium chloride (BAK).

Under certain circumstances, a surfactant might be used in any of the compositions related to this invention which are described herein. The term surfactant used herein has the meaning commonly understood in the art. Surfactants are used to help solubilize the therapeutically active agent or other insoluble components of the composition, and may serve other purposes as well. Anionic, cationic, amphoteric, zwitterionic, and nonionic surfactants may all be used in this invention. For the purposes of this invention, it is preferable that a nonionic surfactant, such as polysorbates, poloxamers, alcohol ethoxylates, ethylene glycol-propylene glycol block copolymers, fatty acid amides, alkylphenol ethoxylates, or phospholipids, is used in situations where it is desirable to use a surfactant.

In another embodiment, a chelating agent may be used. The term chelating agent refers to a compound that is capable of complexing a metal, as understood by those of ordinary skill in the chemical art. Chelating agents are used in ophthalmic compositions to enhance preservative effectiveness. While not intending to be limiting, some useful chelating agents for the purposes of this invention are edetate salts, like edetate disodium, edetate calcium disodium, edetate sodium, edetate trisodium, and edetate dipotassium.

### EXAMPLES

The method of the invention is illustrated by the following examples.

The mouse model of dry eye has been a valuable tool for dissecting the immunopathogenic mechanisms of disease. In brief, mice exposed to desiccating stress display rapid and coordinated upregulation of proinflammatory cytokines that precedes autoreactive CD4+ T cell activation. The inventors obtained 6-10 week old, female C57BL/6 (C57BL/6NTac) mice from Taconic, Inc. (Germantown, N.Y.). Animal studies approval was obtained from the Allergan Animal Care and Use Committee. Dry eye was induced by treating mice with subcutaneous injections of scopolamine hydrobromide (0.5 mg/0.2 ml; Sigma-Aldrich, St. Louis, Mo.) three times a day, alternating between the left and right flanks. Mice were placed in a cage containing perforated plastic screens to permit airflow from fans (one fan on each side of the cage) for 16 hr/day in a hood (AirClean Systems, Raleigh, N.C.). Room humidity was kept below 40%. Desiccating stress (DS) was induced for 10 consecutive days. Mice displayed rapid and coordinated upregulation of proinflammatory cytokines that preceded CD4+ T cell activation in the secondary lymphoid organs. By 5 days post-DS a mixed population of infiltrating inflammatory cells including CD4+ T cells (Th1 and Th17) were detected within the ocular surface tissues, along with elevated levels of Th1- and Th17-derived cytokines, IFN-γ and IL-17. Accumulation of T cells and their derivatives correlated with decreased goblet cell density in the conjunctiva, epithelial cell apoptosis, increased corneal permeability and squamous metaplasia of the corneal surface. Furthermore, adoptive transfer of DS-primed CD4+ T cells isolated from mice exposed to DS for 5-10 days caused ALKC in nude recipient mice, even though the nude mice were never exposed to DS. By 3 days-post transfer, the recipient mice exhibited increased proinflammatory cytokine levels in the tears, extensive cellular infiltration into the conjunctiva, cornea and lacrimal gland, decreased tear production and loss of goblet cells. CD4+ T cells were not detected within other organs of nude recipient mice confirming that DS-specific CD4+ T cell homing and pathogenesis was restricted to the ocular surface tissues.

Accumulation of T cells and their derivatives correlates with decreased Goblet cell density in the conjunctiva, epithelial cell apoptosis, increased corneal permeability and squamous metaplasia of the corneal surface (Niederkorn et al, 2006; de Paiva 2007, 2009). While the eitiology in unknown, the inventors hypothesize that environmental stress (e.g. microbial infection, desiccating stress) in the context of a genetic predisposition compromises the tolerance mechanisms i.e. immunoregulation present within the ocular surface tissues to trigger the initial inflammatory events leading to chronic T cell-mediated Dry Eye disease. This model offers the advantage of more precisely dissecting T cell-specific aspects (effector and regulatory) that contribute to the development of, and protection from, ocular surface autoimmunity.

The healthy eye contains a variety of immunoregulatory mechanisms to tightly control the immune response to limit bystander tissue damage following stress and/or microbial assaults. Among the variety of immunoregulatory mechanisms, regulatory cells have been proposed to modulate the immune response within the ocular surface tissues and regional lymphoid organs. In the context of experimental Dry Eye, conventional CD4⁺CD25^{hi}Foxp3⁺ nTregs confer protection from full-blown T cell-mediated experimental autoimmune lacrimal keratoconjunctivitis (ALKC) (Niederkorn et al, 2006). CD4⁺CD25^{hi}Foxp3⁺ cells also mute inflammation when co-transferred with ALKC-specific pathogenic CD4+ T cells into T cell-deficient nude mice (Siemasko et al, 2008), further highlighting the suppressive role of Tregs in ocular surface autoimmunity. To date, the specific mechanisms used by regulatory cells to inhibit T cell activation, differentiation, proliferation and effector function have not been extensively elucidated, but have been proposed to mediate suppression by, for example, releasing soluble factors (e.g. TGF-β, IL-10). Indeed, TGF-β is present on the ocular surface (Gupta et al, 1996) and has profound suppressive affects on resident dendritic cell maturation in the cornea (Shen et al, 2007) autoreactive T cell proliferation, differentiation, and survival (Gorelik et al, 2000) and Treg differentiation and maintenance (Horwitz et al, 2003; Chen et al, 2003).

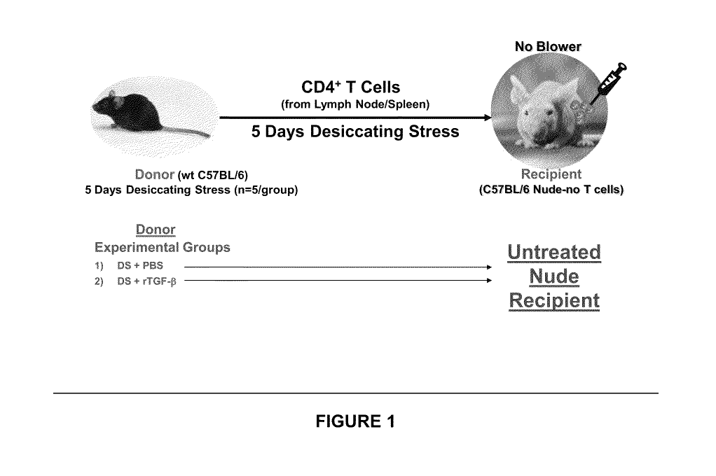

The inventors found that topical treatment with recombinant TGF-β attenuated activation of pathogenic T cells and inhibited the development of experiemental Dry Eye. Mice exposed to desiccating stress were treated topically with recombinant mouse TGF-β (100 ng/5 μl/eye/BID); following 5 days of stress CD4+ T cells were isolated and adoptively transferred to nude recipient mice to assess autoreactive T cell activation. The procedure is illustrated in FIG. 1.

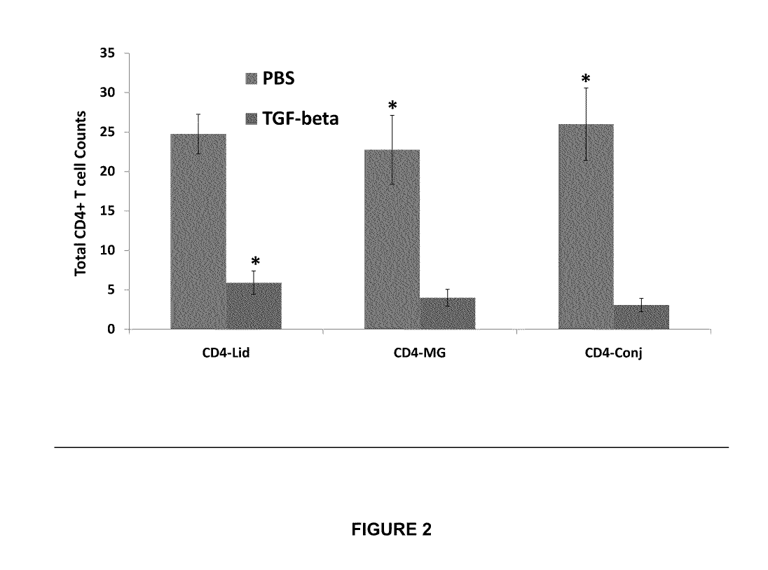

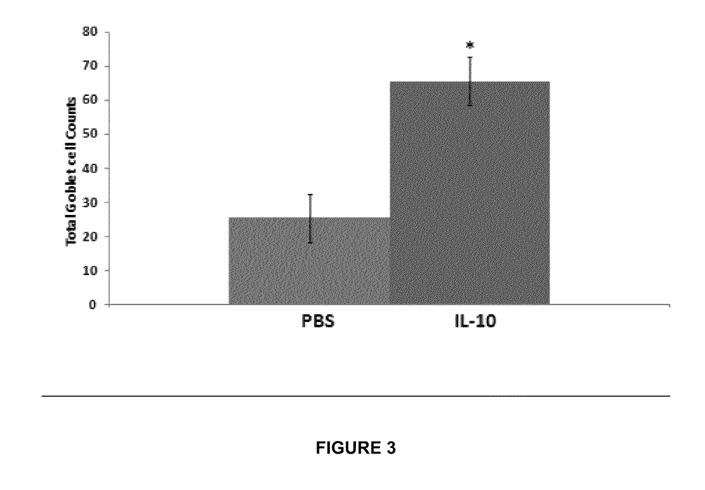

Nude recipients of CD4+ T cells from mice exposed to desiccating stress in the presence of rTGF-β displayed a significant (p≦0.05) reduction in the number of infiltrating CD4+ T cells within the ocular surface tissues (Lid, meibomian gland—MG, conjunctiva—conj) (Lid: 5.9±1.5, MG: 4.0±1.1, conj: 3.1±0.8) compared to mice treated with vehicle alone (Lid: 24.8±2.5, MG: 22.8±4.4, conj: 26±4.6) (FIG. 2). Furthermore, these mice displayed a significant (p≦0.05) preservation of the total number of Goblet cells within the conjunctiva (65.7±8.8); by contrast, mice receiving vehicle alone displayed a dramatic loss of Goblet cells (25.6±8.7) (FIG. 3). Collectively, these data indicate that topical application of full length recombinant TGF-β inhibits generation of autoreactive CD4+ T cells and preserves the integrity of the ocular surface tissues during the development of experimental Dry Eye and provides evidence that this approach is a viable therapeutic strategy to reduce the signs and symptoms of chronic T cell-mediated Dry Eye in humans.

What is claimed is:

1. A method for the treatment of an ocular condition selected from keratoconjunctivitis sicca, diminished corneal sensitivity, atopic keratoconjunctivitis, vernal keratoconjunctivitis, ocular cicatricial pemphigoid, or blepharitis, the method comprising administering to a patient having the condition a composition comprising a TGF-β.

2. The method of claim 1, wherein the condition is keratoconjunctivitis sicca.

3. The method of claim 1, wherein the condition is diminished corneal sensitivity.

4. The method of claim 1, wherein the condition is atopic keratoconjunctivitis

5. The method of claim 1, wherein the condition is vernal keratoconjunctivitis.

6. The method of claim 1, wherein the condition is ocular cicatricial pemphigoid.

7. The method of claim 1, wherein the condition is blepharitis.

8. The method of claim 1, wherein the TGF-β is TGF-β1, TGF-β2, TGF-β3, TGF-β4, or TGF-β5.

9. The method of claim 2, wherein the TGF-β is TGF-β1, TGF-β2, TGF-β3, TGF-β4, or TGF-β5.

10. The method of claim 3, wherein the TGF-β is TGF-β1, TGF-β2, TGF-β3, TGF-β4, or TGF-β5.

11. The method of claim 4, wherein the TGF-β is TGF-β1, TGF-β2, TGF-β3, TGF-β4, or TGF-β5.

12. The method of claim 5, wherein the TGF-β is TGF-β1, TGF-β2, TGF-β3, TGF-β4, or TGF-β5.

13. The method of claim 6, wherein the TGF-β is TGF-β1, TGF-β2, TGF-β3, TGF-β4, or TGF-β5.

14. The method of claim 7, wherein the TGF-β is TGF-β1, TGF-β2, TGF-β3, TGF-β4, or TGF-β5.

15. A method for the treatment of a patient having Stevens-Johnson disease or graft versus host disease, the method comprising administering to the patient a TGF-β.

16. The method of claim 15, wherein the patient has Stevens-Johnson disease.

17. The method of claim 15, wherein the patient has graft versus host disease.

18. The method of claim 15, wherein the TGF-β is TGF-β1, TGF-β2, TGF-β3, TGF-β4, or TGF-β5.
